(19) United States
(12) Patent Application Publication
(10) Pub. No.: US 2008/0154213 A1
(43) Pub. Date: Jun. 26, 2008

(54) Syringe with multiple plunger locks

(76) Inventor: Bruce Leigh Kiehne, Springwood (AU)

Correspondence Address: Hoffman, Wasson & Gitler, P.C., Suite 522, 2461 South Clark Street, Arlington, VA, 22202, US

(21) Appl. No.: 11/653,317

(22) Filed: Jan. 16, 2007

(30) Foreign Application Priority Data
Dec. 21, 2006 (AU) 2006907179

(51) Int. Cl. A61M 5/315

(52) U.S. Cl. 604/220

(57) ABSTRACT

A syringe having a syringe body, a plunger, means to prevent the rear of the plunger from being gripped to retract the plunger when the plunger is at least at the fully forward position where all or most of the liquid has been ejected, and a catch on the plunger which prevents retraction of the plunger when the plunger is in at least the fully forward position.

### FIELD OF THE INVENTION

This invention is directed to a syringe, and typically to a safety syringe which has a particular design to lock the plunger at multiple different points. As an example, the design may be such that the plunger can be locked against total withdrawal from the syringe barrel, and can be locked when the plunger is in the forward (depressed) position. If the syringe has a needle retraction mechanism, the design of the syringe may also be such that the plunger can be locked against retraction when all the liquid in the syringe has been expelled but prior to triggering of the retraction mechanism.

### BACKGROUND OF THE INVENTION

The risk of needlestick injury, particularly with medical syringes has resulted in a number of safety syringes being developed. The applicant is a pioneer in the field of such safety syringes. A safety syringe may be of the type where the needle can be retracted into the safety of the syringe body after use. One main type of safety syringe comprises some form of shoot back mechanism where a spring or something similar is provided in a forward part of the syringe body and which is under compression and, when the plunger is pushed fully into the syringe body and against a needle holder, the spring is allowed to expand which pushes the needle holder+the needle back into the syringe body or the plunger. Applicant has earlier patent applications/patents which describe a particular type of shoot back mechanism. Another main type of safety syringe comprises some form of coupling between the plunger and the needle holder when the plunger is pushed fully forward in the barrel. The plunger couples to the needle holder and retraction of the plunger causes the needle to be retracted.

The design of the present invention finds particulars suitability with a safety syringe of the type that has a needle holder which can be retracted by some form of triggering action and typically using a spring or something similar. However, the present invention, or parts of the present invention may also find suitability with other types of syringes and similar needle containing medical devices.

With syringes, one disadvantage with the design of the syringe is that the plunger can often be pulled out (retracted) too far such that the plunger is pulled entirely out of the syringe body. Sometimes, it is difficult to reinsert the plunger back into the syringe body. Other times, there can be damage to the plunger (and particularly to the plunger seal) which makes the syringe useless. When drawing up liquid into the syringe the plunger is retracted to create a partial vacuum in the syringe body to draw the liquid into the syringe body. It is possible to retract the plunger too far causing the plunger to be pulled entirely out of the syringe body with the liquid spilling out through the rear of the syringe body.

For this reason, it is known to provide some form of mechanism or design that prevents the plunger from being pulled entirely out of the syringe body. A disadvantage with these plunger lock mechanisms is that the plunger sometimes needs to be partially inserted into the syringe body prior to providing the lock mechanism. This can create manufacturing difficulties.

With safety syringes of the type that have a shoot back mechanism, there is a manufacturing advantage in being able to insert the needle holder through the open rear end of the syringe barrel and then to push the needle holder into position into the front of the syringe barrel. This can be called “rear assembly”. This is in contrast to other safety syringes where the needle holder is assembled through the front of the syringe body which is not particularly desirable.

A disadvantage with some existing plunger lock mechanisms of the type that prevent the plunger from being fully retracted out of the syringe body is that the mechanism may also interfere with the rear assembly of the needle holder. That is, the plunger lock mechanism can have parts which prevent the needle holder from being pushed to the front of the barrel without possible damage to the needle holder. To explain, one type of needle holder comprises a break web or frangible portion as part of the needle holder which is designed to break during the retraction mechanism. Any damage of this part of the needle holder by conflict with the plunger lock mechanism may cause the triggering action to be unreliable.

There is an advantage in being able to lock or hold the plunger when the plunger is in the forward (depressed) position. There is also an advantage in having a “forward” type of plunger lock mechanism which locks the plunger when the plunger is in the front of the syringe.

With safety syringes of the type that have a retracting (typically shoot back) needle holder there is an issue in ensuring that all the medicine/liquid in the syringe is expelled before the retraction mechanism is triggered. Put differently, it is not considered desirable to have an appreciable amount of “dead space” in the syringe.

The applicant's earlier patent applications describe a design that leaves virtually no dead space in the syringe and therefore provides distinct benefits to the safety syringe. That is, the applicant's syringe provides for a reliable shoot back mechanism as well as very little or no dead space.

There may be other syringe designs that also overcome the issue of dead space prior to triggering the retraction mechanism.

Therefore, with these particular designs it is possible, by careful manipulation of the plunger, to push the plunger almost fully forward to eject all the liquid in the syringe but to stop the plunger just prior to the triggering point. A disadvantage with this arrangement is that the plunger can now be retracted and this syringe can technically be reused which may cause contamination.

Therefore, there would be an advantage if it were possible to have a plunger lock arrangement that could lock the plunger against retraction when the plunger is in the position where all or almost all the liquid has been ejected but just prior to the trigger point. This position can be called the “zero mark” position.

Many single use syringes having the shoot back mechanism rely upon the plunger being pushed against part of the needle holder or some other part in the forward part of the syringe, to trigger the retraction sequence. For instance, in the applicant's earlier syringe design, the needle holder comprises a first part (outer part) which is attached to, or supported by, a front portion of the syringe barrel and a second part (inner part) which contains the needle and the spring. When the plunger is pushed fully forwardly into the syringe body, the front of the plunger breaks the outer parts from the inner part and the spring then shoots the inner part (containing the contaminated needle) back into the hollow plunger body.

Although at this stage, the needle is relatively safe in the plunger body, there would be an advantage if a plunger lock mechanism could be provided which would prevent retraction of the plunger after the needle retraction mechanism has triggered such that the situation could not occur with the plunger could be removed and the needle removed from the plunger for possible reuse.

It will be clearly understood that, if a prior art publication is referred to herein, this reference does not constitute an admission that the publication forms part of the common general knowledge in the art in Australia or in any other country.

### BRIEF SUMMARY OF THE INVENTION

It is an object of the invention to provide a needle containing medical device and particularly a needle containing medical device having a plunger and where the plunger is able to be locked against certain types of movement at multiple positions.

It is a further object of the invention to provide a needle containing medical device which may overcome at least some of the above-mentioned disadvantages or provide a useful or commercial choice.

In one form, the invention resides in a syringe having a syringe body, the syringe body having a forward end, and an open rear end, a plunger which is movable along the syringe body, the plunger having a plunger body, a plunger seal in a forward part of the plunger body, and a thumb pad in a rear part of the plunger body, a needle holder which is in/on the forward end of the syringe body, a needle which is attached to the needle holder, and a rear plunger lock which comprises at least one deflectable finger supported by the syringe body and which extends at least partially into the syringe body, the plunger having a catch in a forward part of the plunger, the catch engaging with the at least one deflectable finger when the plunger is sufficiently retracted, the catch and deflectable finger preventing the plunger from being fully retracted out of the syringe body.

Thus, during the manufacturing process, the plunger can be initially inserted through the open rear of this syringe body and the catch will push away the deflectable finger without damage to the finger or the plunger. However, once the plunger has been inserted, the design of the deflectable finger and the catch is such that the plunger can not be removed from the syringe body.

The needle holder can also be inserted from the rear of the syringe body, without being damaged by the deflectable finger.

It is envisaged that a pair of deflectable fingers will be provided and it is envisaged that these will be at diametrically opposed parts of the syringe body. There may be circumstances where it may be desirable to have more than a pair of deflectable fingers but it is considered that for syringes of between 1-10 mils, a pair of deflectable fingers will be sufficient.

The position of the at least one deflectable finger should be such that the plunger cannot be entirely removed from the syringe body but where the plunger can still sufficiently retract to draw liquid into the syringe body. Usually, this means that the at least one deflectable finger will be positioned in a rear part of the syringe barrel and typically just in front of the laterally extending “wings” which almost all syringes have.

The at least one deflectable finger may comprise a “cantilevered” rectangular portion which is attached to the syringe barrel at a rear portion and is cantilevered towards the front portion of the syringe. The size of the deflectable finger may vary but it is considered that suitable size will be a length of between 3-20 mm and a width of between 3-10 mm. The deflectable finger will typically be substantially rectangular but it may also be of other shapes such as oval, part circular and the like.

The cantilevered finger is designed to extend a small way into the syringe barrel and to be naturally biases in this position. It is envisaged that the finger will deflect into the syringe barrel by a distance of between 0.5-5 mm and typically between 1-2 mm.

The at least one deflectable finger is designed to catch against a catch on the plunger and to prevent the plunger from being further retracted. In a simple design, the catch on the plunger may comprise some form of extension and may comprise a collar which extends about the plunger such that the collar will catch against the deflectable finger irrespective of any rotation of the plunger relative to the barrel. The collar may comprise a disk like structure and the positioning of the collar (catch) should be such that it engages with the at least one deflectable finger when the plunger is sufficiently retracted to allow liquid to be drawn into the syringe body but preventing the plunger from being pulled entirely out of the syringe body. For this reason, it is preferred that the catch on the plunger is positioned in a forward part of the plunger body and preferably closely behind the plunger seal.

The catch may have other designs. For instance, the catch may comprise a series of projections extending from the plunger body and designed to engage with the at least one deflectable finger.

The syringe may have any convenient shape and size and it is envisaged that the syringe will hold an injectable volume of between 1-20 ml although this can vary.

The plunger body may be substantially hollow to accommodate the retracted needle+needle holder. However it is also envisaged that the plunger body has an X type cross-section and the needle+needle holder can pass into a cavity between the plunger body and the syringe body.

The plunger will typically have a thumb pad on the rear of the plunger and these thumb pads are quite conventional and well-known. The term “thumb pad” is meant to include any design on the rear of the plunger can facilitate pushing of the plunger, typically by a persons thumb.

The needle holder may comprise a design described in one or more of our earlier patent applications but may also comprise a design described in other patent applications or in other commercial products or published products. Usually, the needle holder will contain some part which can contain the needle and this part is usually a central part or inner part of the needle holder. The needle holder or part of the needle holder will typically be held in some releasable manner in a forward part of the syringe body and can be “triggered” or otherwise manipulated to cause retraction of the needle. In our earlier patent applications, the needle holder is held in a front part of the syringe body by a step or something similar. In other designs, the needle holder is held in the front part of the syringe body by other means including friction, a shatter ring and the like.

Part of the needle holder is typically biased by spring or something similar to shoot back into the plunger body/syringe body but is prevented from doing so by being held in some manner and this may comprise a frangible portion and the like.

The triggering action to release part of the needle holder (containing the needle) for retraction usually occurs by part of the plunger contacting part of the needle holder when the plunger has been pushed in the fully forward position.

These mechanisms of retraction are described in some of our earlier patent applications and in other publications.

In another form the invention resides in a syringe which may have at least some of the parts mentioned previously and including a plunger lock mechanism which prevents retraction of the plunger when all, or substantially all of the liquid in the syringe has been expelled (zero mark) but prior to triggering of a new needle retraction mechanism.

In this form of the invention, the plunger lock mechanism may be separate, or in addition to, the mechanism described previously which prevents the plunger from being pulled entirely out of the syringe or may be separate.

Thus, in this form of the invention (which may conveniently be called the zero mark plunger lock), a syringe may comprise only the zero mark plunger lock or the zero mark plunger lock together with the plunger lock described previously.

The zero mark plunger lock may comprise any type of an anti retraction mechanism which operates when the plunger is at the or close to the zero mark position.

Suitably, the zero mark plunger lock comprises at least one deflectable finger supported by the syringe body and a catch in a rear part of the plunger, the catch engaging with the at least one deflectable finger to prevent retraction of the plunger when the plunger is in the zero mark position.

It is preferred that the at least one deflectable finger is similar to that described previously, and it is particularly preferred that the at least one deflectable finger on the zero mark plunger lock is the same as the deflectable finger which prevents the plunger from being entirely retracted from the plunger body. Thus, this deflectable finger can be “dual purpose”.

The catch on the zero mark plunger lock may have a configuration which is similar to that described above and therefore may comprise a collar, a disk, or one or more projections which engage with the least one deflectable finger. This catch however is preferably positioned in a rear portion of the plunger such that when the plunger is pushed forwardly, the catch will pass underneath the at least one deflectable finger just prior to the zero mark position which means that the plunger cannot be retracted once the catch has passed the at least one deflectable finger.

In another form the invention resides in a syringe having a syringe body, a plunger, and means to prevent the rear of the plunger from being gripped to retract the plunger when the plunger is at least at the fully forward position where all or most of the liquid has been ejected. To explain, with a conventional syringe, once the plunger has been pushed forwardly, it is still possible to grip the end of the plunger (typically the thumb pad) to retract the plunger. In this particular form of the invention, the design of the syringe is such that the grippable end of the plunger is protected or covered and cannot be easily gripped.

In this form of the invention the means may comprise a cover member or a shroud or something similar that can extend about the side portion of the end of the plunger when the plunger is pushed into the barrel. As an example, the shroud may comprise a circular collar or something similar which extends about the side portion of the plunger. The collar may comprise a continuous, or a number of projections or something similar such that the edges of the thumb pad cannot be gripped to try to retract the plunger. Another advantage of the cover member or shroud is that once the plunger is in this position a practitioner can obviously see that the syringe has been rendered safe and useless.

The cover member/shroud etc may be provided on an otherwise conventional type syringe. However, it is preferred that the cover member/shroud etc is provided on a safety syringe of the type that has a needle retraction mechanism and therefore the cover member/shroud may be provided on a safety syringe of the type described above.

In an especially preferred form of the invention, there is provided a syringe or other applicable type of needle containing medical device which contains the plunger lock mentioned above which prevent the plunger from being entirely retracted from the needle, and a zero mark plunger lock as described above and a cover member/shroud as described above.

In this especially preferred form of the invention, it is preferred that the retraction mechanism is triggered just prior to the rear end of the plunger being protected by the cover member/shroud etc.

In yet another form of the invention there is provided a syringe having a syringe body, a plunger, means to prevent the rear of the plunger from being gripped to retract the plunger when the plunger is at least at the fully forward position where all or most of the liquid has been ejected, and a catch on the plunger which prevents retraction of the plunger when the plunger is in at least the fully forward position.

In this form of the invention, the syringe may comprise the cover member/shroud as described above and the plunger may be provided with another catch portion which may be similar to that described previously and which engages with the least one deflectable finger should it be possible to retract the plunger when the plunger is in this fully forward position. This form of the invention can be seen as a “double safety” form as it contains the combination of the cover member/shroud which should prevent the end of the plunger from being gripped and retracted but in any event there is provided another catch which engages with the at least one deflectable finger to prevent retraction of the plunger.

It should be appreciated that the disclosure of any publication does not mean that the disclosure forms part of the common general knowledge in Australia or elsewhere.

### BRIEF DESCRIPTION OF THE DRAWINGS

An embodiment of the invention will be described with reference to the following drawings in which:

FIG. 1. Illustrates a syringe according to an embodiment of the invention and which contains a rear plunger lock, a forward (zero mark) plunger lock and means (such as a cover member or a shroud) to extend about a rear portion of the plunger when the plunger is pushed for the forwardly.

FIG. 2. Illustrates a section view of FIG. 1 and illustrates the position of the plunger just prior to the zero mark plunger lock configuration.

FIG. 3. Illustrates a section view of the syringe with the plunger in the fully retracted position and illustrating the working of the rear plunger lock that prevents the plunger from being pulled out of the syringe body.

FIG. 4. Illustrates movement of the plunger from the fully retracted position of FIG. 3 and towards the zero mark position.

FIG. 5. Illustrates the forward zero mark plunger lock position where the plunger is locked against retraction but the needle retraction mechanism has not yet been triggered.

FIG. 6. Illustrates the triggered position where the needle holder+the needle has been retracted into the plunger body and the rear end (thumb pad) of the plunger body is within a protective shroud and therefore cannot be gripped and retracted.

FIG. 7. Illustrates a further locking means which engages against the deflectable finger when the syringe is in the triggered position and to provide yet a further lock to prevent retraction of the plunger.

### DETAILED DESCRIPTION

Referring to the drawings there is illustrated a syringe which contains a number of plunger lock mechanisms and although the embodiment refers to a syringe containing the combination of the plunger lock mechanisms, it should be appreciated that the invention may also extend to a syringe containing only one or any other combination of the plunger lock mechanisms.

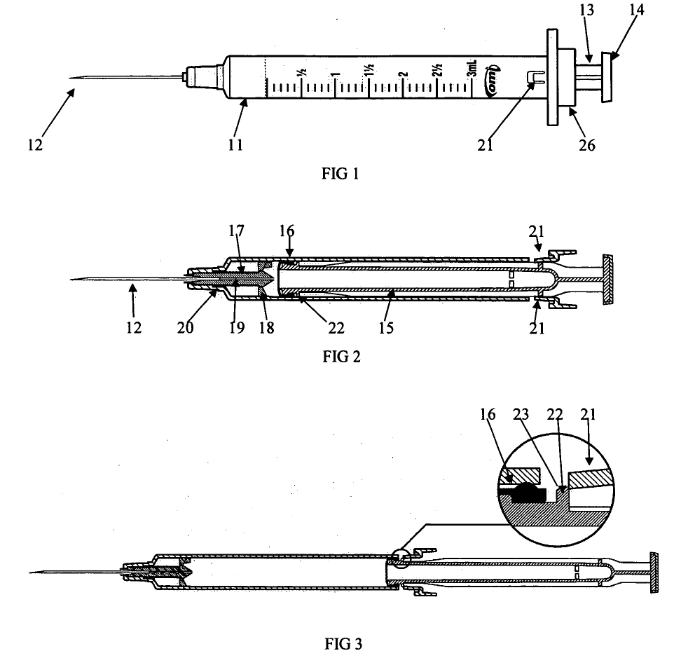

Referring to the drawings and initially to FIG. 1, there is illustrated a syringe 10 having a body 11 a needle 12 extending from the front of the syringe body 11 and a plunger 13 which has a rear thumb pad 14 which is circular. Referring to FIG. 2, plunger 13 has a plunger body 15 and a plunger seal 16 in the front of plunger body 15. The plunger 13 is hollow to accommodate the retracted needle is will be described in greater detail below.

A needle holder 17 is provided and to which needle 12 is attached. Needle holder 17 in the particular embodiment, comprises a design described in one or more of our earlier published patent applications and in brief, needle holder 17 contains an outer portion 18 and an inner portion 19. Inner portion 19 supports a small compressed spring 20. Inner portion 19 and outer portion 18 are attached by in such a way that the inner portion can break away from the outer portion. The needle retraction (triggering) mechanism occurs when the front of plunger 13 is pushed against the needle holder 17 and when this occurs the plunger pushes the outer portion 18 preferentially which causes the outer portion to break away from the inner portion 19. As soon as this occurs, the compressed spring 20 expands to shoot the inner portion 19 of the needle holder+the attached needle 12 into the plunger body as illustrated in FIG. 6 and FIG. 7 the outer portion 18 remains in the front of the syringe barrel.

The syringe 10 contains a rear plunger lock mechanism that prevents the plunger 13 from being pulled entirely out of the syringe body 11. This rear plunger lock mechanism is best described and illustrated with reference to FIGS. 1-3. In particular, the rear plunger lock mechanism comprises a pair of deflectable fingers 21 which are positioned in a diametrically opposed manner on the rear end of plunger body 11. FIG. 1 illustrates only one deflectable finger 21 while FIG. 2 illustrates (in section) both deflectable fingers 21. Each deflectable finger comprises a small somewhat rectangular member which is attached at a rear portion to the syringe body and which extends in a cantilevered manner towards the front of the syringe body. The deflectable finger naturally adopts a slightly inwardly bent configuration such that it projects slightly into the syringe barrel. This slight inwardly bent configuration is illustrated in FIG. 2.

In the initial assembly process, the plunger 13 can be inserted through the open rear end of the body and pushed partially into their syringe body. During this process, the plunger will push past the deflectable fingers and the fingers will momentarily deflect outwardly to allow the front of the plunger to pass. Immediately afterwards, the fingers will deflect back to their slightly inwardly bent configurations.

The forward part of the plunger has a catch 22 immediately behind seal 16 and in the particular embodiment, catch 22 comprises a circular flange or disk on the plunger body. Part of the catch 22 is illustrated in FIG. 3.

Thus, once the plunger has been initially inserted into the syringe barrel (e.g. the position illustrated in FIG. 2), the plunger can be retracted in the usual manner to draw liquid into their syringe body but for the retraction will cause catch 22 to catch against the end of the deflectable fingers 21 (see FIG. 3) which will prevent further retraction of the plunger.

The front of the catch has a small ramped portion 23 (see FIG. 3) which assists in the initial insertion of the plunger into the syringe body by assisting in the momentary deflection of finger members 21.

Thus, the plunger lock assembly as described above can be seen as a “rear plunger lock” which prevents the plunger from being fully retracted out of the barrel.

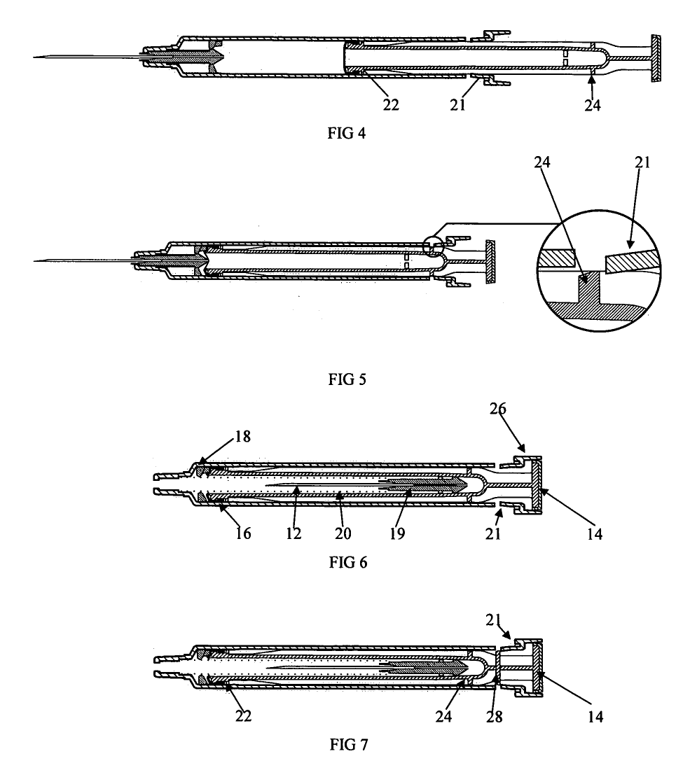

The syringe according to the preferred embodiments also has a “zero mark plunger lock” which is best illustrated in FIG. 5. In the particular embodiment, the “zero mark” is the position that the plunger has where all, or substantially all of the fluid has been ejected but the retraction mechanism has not yet been triggered. FIG. 5 illustrates this position where the front of the plunger (e.g. seal 16) is against the needle holder 17 but has not yet been pushed further forwardly to trigger the retraction mechanism. To prevent the plunger from being retracted at this “zero mark” position, there is provided a “zero mark plunger lock”. In the preferred embodiment illustrated in FIG. 5, the plunger body 15 is provided with another catch in the form of a circular disc or collar or projection 24 (see FIG. 5). This catch 24 is positioned in a rear part of the plunger and catch 24 is also illustrated in FIG. 4. When the plunger moves towards the zero mark position, catch 24 (which again has a small ramped forward face) pushes underneath and deflects fingers 21 momentarily and is being positioned in front of fingers 21. In this position, the plunger cannot be retracted as catch 24 will engage with fingers 21.

The syringe according to the preferred embodiment contains a further safety feature to prevent retraction of the plunger when the retraction mechanism has been triggered. This further safety feature is illustrated in FIG. 6 and comprises a circular shroud 26 on the rear of syringe body 11 (shroud 26 is also illustrated in FIG. 1). The length of plunger 13 is designed such that when the plunger has been pushed fully forwardly against needle holder 17 to trigger the retraction mechanism, the thumb pad 14 or other end of the plunger 13 sits within shroud 26 (see FIG. 6 and FIG. 7), and it can be appreciated that the thumb pad 14 is now almost impossible to grip to try to retract the plunger. If the thumb pad is oval or a different shape, the shroud will have a corresponding shape to snugly cover the edges of the plunger.

However, to provide even further security, there may be provided a further plunger lock mechanism which is illustrated in FIG. 7 and which comprises yet another catch 28 which is behind catch 24 and which is designed to engage against the deflectable fingers 21 when the plunger thumb pad is protected by shroud 26. Thus, even if it were possible to grip the rear parts of plunger 26, any retraction would cause catch 28 to lock against fingers 21, and if this catch failed, catch 24 would lock against fingers 21.

Throughout the specification and the claims (if present), unless the context requires otherwise, the term “comprise”, or variations such as “comprises” or “comprising”, will be understood to apply the inclusion of the stated integer or group of integers but not the exclusion of any other integer or group of integers.

Throughout the specification and claims (if present), unless the context requires otherwise, the term “substantially” or “about” will be understood to not be limited to the value for the range qualified by the terms.

Any embodiment of the invention is meant to be illustrative only and is not meant to be limiting to the invention. Therefore, it should be appreciated that various other changes and modifications can be made to any embodiment described without departing from the spirit and scope of the invention.

1. A syringe having:
a syringe body having a forward end and an open rear end,
a plunger which is movable along the syringe body, the plunger having a plunger body, a plunger seal in a forward part of the plunger body, and a thumb pad in a rear part of the plunger body,
a needle holder which is in the forward end of the syringe body,
a needle which is attached to the needle holder, and,
a rear plunger lock which comprises at least one deflectable finger supported by the syringe body and which extends at least partially into the syringe body, the plunger having a catch in a forward part of the plunger, the catch engaging with the at least one deflectable finger when the plunger is sufficiently retracted, the catch and deflectable finger preventing the plunger from being fully retracted out of the syringe body.

2. The syringe of claim 1, comprising at least two said deflectable fingers positioned in a rear part of the syringe barrel.

3. The syringe of claim 2, wherein each deflectable finger comprises a cantilevered rectangular portion which is attached to the syringe barrel at a rear portion and is cantilevered towards the front portion of the syringe, and which has a length of between 3-20 mm and a width of between 3-10 mm.

4. The syringe of claim 2, wherein the catch on the plunger comprises a collar which extends about the plunger such that the collar will catch against the deflectable finger irrespective of any rotation of the plunger relative to the barrel.

5. The syringe of claim 4, wherein the collar is positioned close to and behind the plunger seal.

6. The syringe of claim 1, wherein the needle can be retracted after use.

7. The syringe of claim 6, wherein the needle holder is releasably held in the front of the syringe barrel and is biased to retract into the syringe body or the plunger, and a triggering means which is activated by the plunger and which allows retraction of the needle holder.

8. The syringe of claim 7 containing a second plunger lock mechanism which prevents retraction of the plunger when all, or substantially all of the liquid in the syringe has been expelled (zero mark position) but prior to triggering of the needle retraction mechanism.

9. The syringe of claim 8, wherein the second plunger lock mechanism includes a catch in the rear part of the plunger, the catch adapted to engage with the at least one deflectable finger to prevent retraction of the plunger when the plunger is in the zero mark position.

10. The syringe of claim 9, wherein the catch is a collar positioned in a rear portion of the plunger such that when the plunger is pushed forwardly, the collar passes underneath the at least one deflectable finger just prior to the zero mark position whereby the plunger cannot be retracted once the catch has passed the at least one deflectable finger.

11. The syringe of claim 1, further including cover means to prevent the rear of the plunger from being gripped to retract the plunger when the plunger is at least at the fully forward position where all or most of the liquid has been ejected.

12. The syringe of claim 11, wherein the cover means is a wall extending about a side portion of the end of the plunger when the plunger is pushed into the barrel, thereby preventing the rear of the plunger from being gripped to retract the plunger.

13. The syringe of claim 8 further including cover means to prevent the rear of the plunger from being gripped to retract the plunger when the plunger is at least at the fully forward position where all or most of the liquid has been ejected.

14. The syringe of claim 13, wherein the cover means is a wall extending about a side portion of the end of the plunger when the plunger is pushed into the barrel, thereby preventing the rear of the plunger from being gripped to retract the plunger.

15. The syringe of claim 11, comprising a further locking catch on the plunger which prevents retraction of the plunger when the plunger is in at least the fully forward position.

16. A syringe having a syringe body, a plunger, means to prevent the rear of the plunger from being gripped to retract the plunger when the plunger is at least at the fully forward position where all or most of the liquid has been ejected, and a catch on the plunger which prevents retraction of the plunger when the plunger is in at least the fully forward position.

17. A syringe having a syringe body, a plunger, and means to prevent the rear of the plunger from being gripped to retract the plunger when the plunger is at least at the fully forward position where all or most of the liquid has been ejected.
